(19) United States
(12) Patent Application Publication
(10) Pub. No.: US 2005/0027317 A1
(43) Pub. Date: Feb. 3, 2005

(54) Defibrillation system for non-medical environments

(76) Inventor: Alois A. Langer, Harrison City, PA (US)

Correspondence Address: Schnader Harrison Segal & Lewis LLP;Suite 3600, 1600 Market Street, Philadelphia, PA, 19103, US

(21) Appl. No.: 10/764,106

(22) Filed: Jan. 23, 2004

Related U.S. Application Data
(60) Provisional application No. 60/442,330, filed on Jan. 27, 2003.

(51) Int. Cl. A61N 1/39

(52) U.S. Cl. 607/5

(57) ABSTRACT

A defibrillation system having a physiological parameter measuring device for measuring parameters that may indicate a need for a defibrillator; an emergency level detector in communication with the physiological parameter measuring device for detecting emergency level physiological parameters; and a notification device in communication with the emergency level detector for providing notice of the detection of an emergency-level physiological parameter to a caregiver who can then utilize a defibrillator.

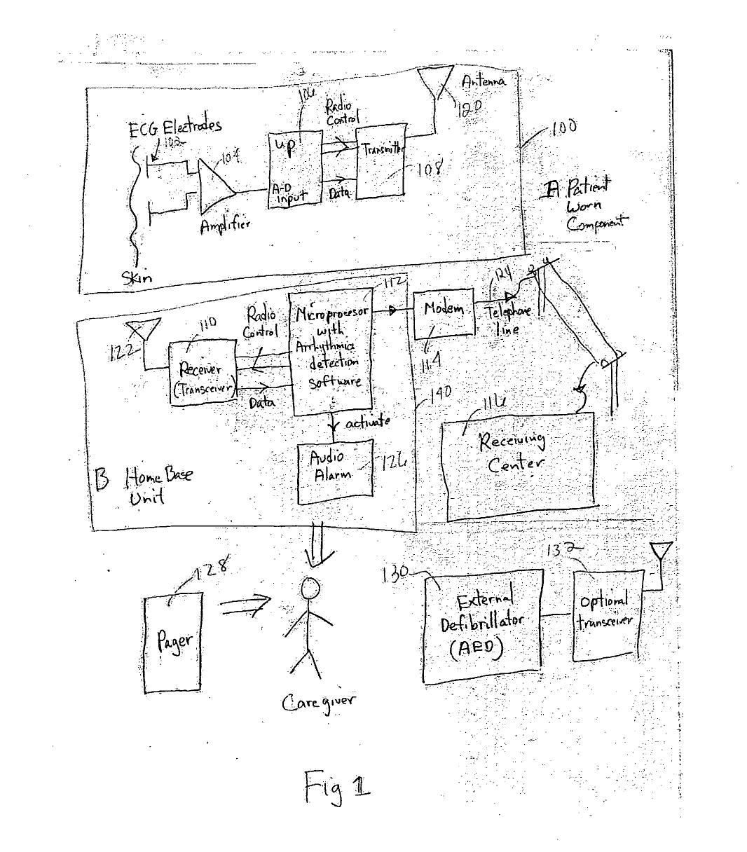

This application is based, and claims priority to, provisional application having Ser. No. ______ having a filing date ______ of entitled Defibrillation System for Home Use.

### BACKGROUND OF THE INVENTION

So-called malignant arrhythmias such as ventricular fibrillation (VF) and sustained ventricular tachycardia (VT) are responsible for a large portion of Cardiac Deaths. Various devices have been developed to deal with this problem in non-medical environments; however, no single device is suitable for all patients. Some devices are implantable and suitable only for patients with a well-established and permanent need for such a device, while others are worn externally.

Microprocessor controlled implantable defibrillators are available, which are capable of automatically terminating various malignant arrhythmias. These devices are in widespread production and have revolutionized the treatment of VF and VT and have saved many thousands of lives. Unfortunately, these devices and the required surgery are expensive, and therefore, the application is limited to permanent implantation. As such these devices are unsuitable for patients who are not hospitalized and are at a temporary increased risk of developing VF or sustained VT such as perhaps patients waiting for a heart transplant.

There also exist patient worn defibrillators, which can be worn temporarily. Though they are suitable for short-term use, they have the disadvantage of being somewhat bulky and heavy and require the wearing of a large vest which can become perspiration soaked and uncomfortable.

There is a class of defibrillators called AEDs or Automatic External Defibrillators, which have been designed to be used in non-medical environments and have even been suggested to be used in at home. These are small external defibrillators which were initially designed to be kept at public places such as airports or in airplanes themselves. If a patient goes into a dangerous heart rhythm and collapses, an observer fetches the defibrillator from it storage place, applies it to the patient and then the defibrillator performs the defibrillation with varying degrees of automaticity. When used at home, these would not have the burden of being continuously worn nor have the permanence of surgical implantation.

The disadvantage of AEDs currently on the market is that they require the response of a person other than the patient. Therefore, these AEDs are ineffective in a situation where another person is unaware of a patient's need for a defibrillator.

Accordingly, there is a need for a defibrillation system that can be used in non-medical environments, when there is no individual immediately aware of the emergency.

### SUMMARY OF THE INVENTION

Embodiments of the present inventive defibrillation system provide notification to a caregiver when a situation arises in which application of a defibrillator is advisable, such as the occurrence of malignant arrhythmias, thus allowing the timely lifesaving application of an AED.

An illustrative embodiment of the invention includes a physiological parameter measuring device for measuring parameters that may indicate a need for a defibrillator;
an emergency level detector in communication with the physiological parameter measuring device for detecting an emergency level in the physiological parameters specific to those requiring defibrillation or cardioversion; and a notification device in communication with the emergency level detector for providing notice of the detection of an emergency-level physiological parameter to a caregiver who can then utilize a defibrillator. The system will preferably be adapted for non-medical use by being small and portable, will be dedicated to a single patient, have backup power in case of a power outage, and have a caregiver notification device capable of notifying the caregiver in a variety of non-medical environments.

### DESCRIPTION OF THE DRAWING

The invention is best understood from the following detailed description when read with the accompanying drawing.

The FIGURE depicts a defibrillation system according to an illustrative embodiment of the invention.

### DESCRIPTION OF THE INVENTION

The present invention is an alarm system that may be used in a non-medical environment, such as in a home, and is designed to notify a caregiver when a patient requires defibrillation. In an illustrative embodiment, a patient wears a strap preferably containing long-term ECG electrodes such as capacitively-coupled skin electrodes, though standard electrodes with wires leading to a small encased device could alternatively be used. Capacitive electrodes, if desired, can be used for patient comfort, since the patient likely will wear the device for an extended period of time. These electrodes provide a physiological parameter from the patient, in this illustrative case an ECG. An amplifier raises the ECG level to a point to be compatible with an analog to digital converter connected to a microprocessor. The microprocessor's software includes a ventricular fibrillation detecting algorithm. Other life-threatening rhythms treatable by a defibrillator may also be detected, but for simplicity these will collectively sometimes be called VF and detectable by a VF detector. Ventricular fibrillation detection algorithms for microprocessors are well known in the art, for example as described in U.S. Pat. No. 6,263,238. Upon occurrence of VF as detected by the microprocessor, an alarm is sounded, alerting someone in proximity to the patient, a so-called caregiver, who then applies an external defibrillator and proceeds to defibrillate.

The system as described above has several limitations, first, that a strap containing all the necessary hardware especially the battery and alarm, might be too heavy to be worn comfortably and placing the alarm on the patient would be extremely frightening should the fibrillation detector make a mistake. Therefore, in another embodiment of the invention, the sensor and the arrhythmia detector are separated, and a radio telemetry transmitter sends the patient's ECG to a receiver located somewhere remote from the patient but where it will alert a caregiver. The remote receiver contains the microprocessor and the audio alarm. This also has the advantage of allowing longer battery life in the patient transmitter since the microprocessor is no longer patient worn. Of course other types of alarms can be used as a notification device such as lights and pagers or combinations thereof. Although at present the detector portion should be separated for patient comfort, it is possible that detectors will be made available in a form that would not necessitate separation.

Other physiological parameters can be used to sense malignant rhythms. In another embodiment of the invention, a light emitting diode sends infrared light into the skin while photosensors detect the scattered light. It is well known to those skilled in the art that a waveform proportional to blood flow can be derived from the photosensor in a manner similar to that used in pulse oximeters. See U.S. Pat. No. 4,807,630 for example. This waveform's pulsatile nature will change when malignant arrhythmias occur and can be used to detect them. Of course the optical method can be used alone or in combination with the ECG or even other physiological parameters. It could be used in a completely patient-worn system or in a system where the detection and alarm function are separated by telemetry as discussed previously.

Fibrillation detectors, or other physiological parameter, emergency-level detectors, while accurate, are not perfect and the possibility of false alarms must be considered. In another embodiment of the invention, an extra confirming step is added between the fibrillation detector and the alarm function. In this version of the invention, after fibrillation is detected, the signal corresponding to the sensed physiological parameter(s) is sent to a remote location such as an off-site central receiving station for human review. The personnel at the receiving center evaluate the physiological data to confirm a malignant rhythm. They then send a confirmation signal to the detector-alarm device enabling the alarm to be activated or they perhaps directly send out an alarm to the caregiver, for example using a pager or cell phone. Communication between the patient's location and the receiving center can be by phone as in the inventor's U.S. Pat. No. 5,966,692 or by other communication means such as the Internet.

The FIGURE depicts an illustrative embodiment of a defibrillation system. Box 100 includes illustrative patient-worn components. ECG electrodes 102 provide an analog ECG signal to an amplifier 104. A microprocessor 106 performs an analog to digital conversion on the signal and sends this digitized signal to a radio telemetry transmitter 108. Preferably, the digitized signal is transmitted continuously. Transmitter 108 is preferably a long-range transmitter.

Box 140 shows a second device, which will be referred to as a home base unit, which functions in conjunction with the patient worn components. Home base unit 140 is placed at the patient location and houses a receiver 110 and a second microprocessor 112. A signal transmitted by antenna 120 of the patient worn portion of the system is received by antenna 122 of home base unit 140. Home base unit 140 performs analysis on the ECG signal received. When an arrhythmia requiring defibrillation is sensed, microprocessor 112 activates a modem 114, or other communications device, and sends the ECG data to a receiving center 116 directly or through an Internet service provider. Modem 114, for example, may be of conventional, cable or DSL variety, perhaps with permanent connection to the Internet, or the transmission may also be wireless as through a conventional cellular voice and data network or dedicated wireless data network such as the new 1xEV-DO (Evolution Data Only) wireless data network.

Personnel at the receiving center verify (or not) the rhythm and send an enabling signal back through a phone line 124, or by the wireless connection, activating an audio alarm 126 at the patient's location. At the same time, receiving center 114 sends a page to an optional pager 128 worn by the caregiver. This is easily done for example through SNPP or Simple Network Paging Protocol or perhaps another protocol from the Central Receiving Center over the Internet. The two warning devices alert the caregiver to the need for defibrillation; the caregiver fetches the home external defibrillator 130 and defibrillates the patient. Optionally, the defibrillator has a radio interface 132 and can communicate with the home base unit allowing the central receiving center to control the defibrillator.

The embodiments presented above are those believed to be most suitable for commercial production and use. The invention will now be further described with respect to a basic embodiment and associated additions and variations.

The basic defibrillation system comprises a physiological parameter measuring device, an emergency level detector in communication with the physiological parameter measuring device for detecting emergency level physiological parameters, and a notification device in communication with the emergency level detector for providing notice of the detection of an emergency-level physiological parameter to a caregiver. As used herein a caregiver includes any individual that may respond to a notification such as an alarm or page, and also includes personnel alerted through a central receiving station. A defibrillator is used in conjunction with the defibrillation system and may be sold as part of the system or separately.

The physiological parameter measuring device measures parameters that may be used to determine if an emergency situation has occurred necessitating use of the defibrillator. Examples of physiological parameter measuring devices include, but are not limited to ECGs, blood flow measurement devices, Pulse Ox sensors, respiration sensors, body movement sensors, and others.

Measurements are provided to the emergency-level detector for a determination of whether the measured parameters have reached an emergency level, necessitating use of a defibrillator. The physiological parameter measuring device and the emergency-level detector may be incorporated into a single device or be separate devices in communication with one another either wirelessly or through hard wiring.

Components of the defibrillation system may be configured to communicate with a central receiving station. The central receiving station may receive communications from the notification components of the system, directly from the physiological parameter measuring device or from the emergency-level detector. If the signal is received from the physiological parameter measuring device, a determination of emergency levels is performed at the receiving station. It is also possible that the signal from the physiological parameter measuring device is routed to somewhere other than the central receiving station for analysis of whether an emergency-level has been reached, before being routed to the central receiving station. Regardless of which component or components are in communication with a central receiving station, the receiving station can serve to verify whether defibrillation is needed.

The central receiving station may also be in communication with the defibrillator so that the defibrillator may be remotely operated, calibrated, tested or otherwise adjusted.

Embodiments of the defibrillation system may also include stored patient data. This data may be available for use for example by the emergency level detection device, the physiological parameter measuring device or the central monitoring station. The system would be configured so the patient data is communicated to the proper system component. The stored patient data may include for example, medical data, patient location data, and emergency contact information.

The invention also includes a method of using a defibrillator. The method includes measuring a physiological parameter, providing the physiological parameter measurement to an emergency level detector, determining if the parameter is at an emergency level, activating a notification device if the physiological parameter is at an emergency level. The notification is received by a caregiver who then utilizes a defibrillator. The methods of the invention include use of components or procedures described herein.

While the invention has been described by illustrative embodiments, additional advantages and modifications will occur to those skilled in the art. Therefore, the invention in its broader aspects is not limited to specific details shown and described herein. Modifications, for example, to the physiological parameters measured, emergency-level detection devices and forms of notification, may be made without departing from the spirit and scope of the invention. Accordingly, it is intended that the invention not be limited to the specific illustrative embodiments, but be interpreted within the full spirit and scope of the appended claims and their equivalents.

1. A defibrillation system comprising:
a) a physiological parameter measuring device;
b) an emergency level detector in communication with the physiological parameter measuring device for detecting emergency level physiological parameters; and
c) a notification device in communication with the emergency level detector for providing notice of the detection of an emergency-level physiological parameter to enable a defibrillator to be utilized.

2. The defibrillation system of claim 1 further comprising a defibrillator.

3. The defibrillation system of claim 1 wherein the physiological parameter measuring device generates an electrocardiogram.

4. The defibrillation system of claim 1 wherein the physiological parameter measuring device measures blood flow.

5. The defibrillation system of claim 4 wherein the physiological parameter measuring device measures blood flow by emitting radiation into the patient's skin and detecting the degree of radiation scattering.

6. The defibrillation system of claim 1 further comprising a wireless communication device for communicating a physiological parameter measured by the physiological parameter measuring device to the emergency level detector.

7. The defibrillation system of claim 6 wherein the wireless communication device comprises:
a transmitter to transmit signals from the physiological parameter measuring device; and
a receiver to receive the transmitted signals;
wherein the receiver is in functional communication with the emergency level detector.

8. The defibrillation system of claim 7 wherein the wireless communication device includes a radio telemetry channel.

9. The defibrillation system of claim 1 wherein the notification device comprises one or more of the following: an audio alarm, a pager and a modem.

10. The defibrillation system of claim 1 further comprising an amplifier to amplify signals generated by the physiological parameter measuring device.

11. The defibrillation system of claim 1 further comprising an analog to digital converter to convert an analog signal from the physiological parameter measuring device to a digital signal.

12. The defibrillation system of claim 1 further comprising a communication device to communicate information from the emergency-level detector, the notification device, or a combination thereof, to a central receiving station.

13. The defibrillation system of claim 2 further comprising a communication device to communicate information from the emergency-level detector, the notification device, the defibrillator, or a combination thereof, to a central receiving station.

14. The defibrillation system of claim 1 wherein the emergency-level detector is a ventricular fibrillation detector.

15. The defibrillation system of claim 1 further comprising an emergency level verification system.

16. The defibrillation system of claim 1 further comprising stored patient data.

17. A method of using a defibrillator comprising:
measuring a physiological parameter;
providing the physiological parameter measurement to an emergency level detector;
determining if the physiological parameter is at an emergency level;
activating a notification device if the physiological parameter is at an emergency level;
receiving a notification from the notification device; and
utilizing a defibrillator to attempt to return the physiological parameter to a non-emergency level.

18. The defibrillation method of claim 17 wherein measuring the physiological parameter comprises creating an electrocardiogram.

19. The defibrillation method of claim 17 measuring the physiological parameter measured is blood flow.

20. The defibrillation method of claim 17 further comprising:
providing the measured physiological parameter to an emergency-level detector using a wireless communication device.

21. The defibrillation method of claim 20 further comprising using a radio telemetry channel.

22. The defibrillation method of claim 17 further comprising:
sounding an audio alarm for notification.

23. The defibrillation method of claim 17 further comprising paging a caregiver.

24. The defibrillation method of claim 17 further comprising amplifying physiological parameter signals.

25. The defibrillation method of claim 17 further comprising converting an analog physiological parameter signal to a digital signal.

26. The defibrillation method of claim 17 further comprising;
providing a signal to a central receiving station if the physiological parameter is at an emergency-level.

27. The defibrillation method of claim 26 further comprising:
operating a defibrillator from the central receiving station.

28. The defibrillation method of claim 17 wherein a ventricular fibrillation detector is used to determine if the physiological parameter is at an emergency-level.

29. The defibrillation method of claim 17 further comprising:
verifying whether the determination of whether the physiological parameter is at an emergency-level is accurate.
